(19) United States
(12) Patent Application Publication
(10) Pub. No.: US 2022/0072216 A1
(43) Pub. Date: Mar. 10, 2022

(54) SHOULDER DRESSING FOR NEGATIVE PRESSURE THERAPY

(71) Applicant: KCI Manufacturing Unlimited Company, Athlone (IE)

(72) Inventors: Jonathan G. Rehbein, San Antonio, TX (US); Richard M. Kazala, San Antonio, TX (US)

(21) Appl. No.: 17/376,821

(22) Filed: Jul. 15, 2021

Related U.S. Application Data
(60) Provisional application No. 63/075,466, filed on Sep. 8, 2020.

(51) Int. Cl. A61M 1/00; A61F 5/058

(52) U.S. Cl. CPC A61M 1/915; A61M 1/917; A61M 2210/086; A61M 2210/083; A61F 5/05858

(57) ABSTRACT

A negative pressure therapy dressing includes a drape layer, an adhesive border configured to provide a seal between the drape layer and skin, and a manifold layer coupled to the drape layer. The manifold layer includes a body portion extending in a first direction, a first wing positioned at a first side of the body portion, and a second wing positioned at a second side of the body portion opposite the first side. The first wing extends away from the first side and partially in the first direction such that a first gap is provided between a first tip of the first wing and the first side of the body portion. The second wing extends away from the second side and partially in the first direction such that a second gap is provided between a second tip of the second wing and the second side of the body portion.

### CROSS-REFERENCE TO RELATED APPLICATIONS

This application claims the benefit of priority to U.S. Provisional Application No. 63/075,466, entitled “SHOULDER DRESSING FOR NEGATIVE PRESSURE THERAPY” filed on Sep. 8, 2020, which is hereby incorporated by reference in its entirety.

### BACKGROUND

The present disclosure relates generally to a wound therapy system, and more particularly to a wound therapy system configured to provide negative pressure wound therapy to the site of one or more shoulder incisions.

Negative pressure wound therapy (NPWT) is a type of wound therapy that involves applying a negative pressure to a wound treatment area to promote wound healing. NPWT can be used to treat wounds in the shoulder area caused by arthroscopic shoulder surgeries. Recent developments in NPWT therapy include the use of adhesive wound dressings that can be positioned over a wound to treat the wound and the surrounding area. However, existing adhesive NPWT dressings are primarily linear dressings designed to treat linear wounds. In most instances, some shoulder surgeries involve three incisions arranged in a non-linear configuration. For example, in some surgical methods, a first incision is made at a front portion of a patient's shoulder, a second incision is made at a back portion of the patient's shoulder, and a third incision is made at a top portion of the patient's arm proximate the patient's shoulder. Conventional NPWT dressings are configured to treat linear incisions and can be time-consuming to modify to treat the specific incision pattern used in arthroscopic shoulder surgeries. Other areas of the human body (and incisions or other wounds thereon) may also be difficult to cover with conventional NPWT dressings.

### SUMMARY

One implementation of the present disclosure a negative pressure therapy dressing. The negative pressure therapy dressing includes a drape layer, an adhesive border configured to provide a seal between the drape layer and skin when the dressing is applied to a patient, and a manifold layer coupled to the drape layer. The manifold layer includes a body portion extending in a first direction, a first wing positioned at a first side of the body portion, and a second wing positioned at a second side of the body portion opposite the first side. The first wing extends away from the first side and partially in the first direction such that a first gap is provided between a first tip of the first wing and the first side of the body portion. The second wing extends away from the second side and partially in the first direction such that a second gap is provided between a second tip of the second wing and the second side of the body portion.

In some embodiments, the adhesive border extends across the gap. The adhesive border may be perforated in the gap to facilitate tearing or cutting of the adhesive border in the gap. The adhesive border may include an adhesive configured to selectively adhere to the skin, the adhesive border, and the drape.

In some embodiments, the manifold layer is scored to facilitate conformability of the manifold layer. The manifold layer may be symmetric across a longitudinal axis of the body portion.

In some embodiments, the body portion includes a concave edge extending between the first wing and the second wing. The first wing may include an elliptical shape. The manifold layer may be configured to be applied to a shoulder region of the patient, with the body portion corresponding to lateral and superior sides of the shoulder region of the patient, the first wing corresponding to an anterior side of the shoulder region of the patient, and the second wing corresponding to a posterior side of the shoulder region of the patient.

Another implementation of the present disclosure is a negative pressure therapy system. The negative pressure therapy system includes a dressing defining a sealable volume and comprising a body portion extending in a first direction and a first wing positioned at a first side of the body portion. The first wing extends away from the first side and partially in the first direction such that a first gap is provided between a first tip of the first wing and the first side of the body portion. The negative pressure therapy system includes a negative pressure source configured to be placed in fluid communication with the dressing and operable to establish a negative pressure at the sealable volume.

The negative pressure therapy system may also include an immobilization device configured to immobilize a shoulder of a patient. The negative pressure source can be coupled to the immobilization device.

In some embodiments, the dressing includes an adhesive border. The adhesive border may extend across the gap. The adhesive border may be perforated in the gap to facilitate cutting or tearing of the adhesive border in the gap.

In some embodiments, the dressing also includes a second wing positioned at a second side of the body portion. The second wing extends away from the first side and partially in the first direction such that a second gap is provided between a second tip of the second wing and the second side of the body portion. The body portion may include a concave edge extending between the first wing and the second wing. The dressing may be symmetrical across a longitudinal axis of the body portion. The first wing may have an elliptical shape.

One implementation of the present disclosure is a method of providing negative pressure therapy. The method includes positioning a body portion of a dressing along a lateral side of a shoulder region of a patient, bending the dressing to position a first wing of the dressing along an anterior side of the shoulder region of the patient, bending the dressing to position a second wing of the dressing along a posterior side of the shoulder region of the patient, adjusting a first gap between the body portion and the first wing and a second gap between the second wing and the body portion to conform the dressing to the shoulder region, establishing a substantially air-tight seal between the dressing and the shoulder region using an adhesive border of the dressing, coupling the dressing to a negative pressure source, and operating the negative pressure source to establish a negative pressure at the dressing and the shoulder region.

In some embodiments, adjusting the first gap includes creating a cut or tear in the adhesive layer proximate the first gap and overlapping the adhesive layer at the cut or tear such that the adhesive layer self-adheres to maintain an adjustment to the first gap. Adjusting the first gap may include decreasing a distance between a tip of the first wing and a first side of the body portion.

In some embodiments, the method also includes immobilizing the shoulder region using an immobilization device and coupling the negative pressure device to the immobilization device.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 illustrates a perspective view of a shoulder area of a representative patient undergoing shoulder surgery.

FIG. 2 is a front view of a wound dressing according to an exemplary embodiment.

FIG. 3 is a perspective view of the wound dressing of FIG. 2 according to an exemplary embodiment.

FIG. 4 is an exploded view of the wound dressing of FIG. 2 according to an exemplary embodiment.

FIG. 5 is a perspective view of a manifold layer of the wound dressing of FIG. 2 according to an exemplary embodiment.

FIG. 6 is an exploded view of a manifold layer of the wound dressing of FIG. 2 according to another exemplary embodiment.

FIG. 7 is a perspective view of the wound dressing of FIG. 2 adhered to a representative person's shoulder.

FIG. 8 is a perspective view of a shoulder dressing, according to an exemplary embodiment.

FIG. 9 is a top view of an embodiment of the shoulder dressing of FIG. 8, according to an exemplary embodiment.

FIG. 10 is a top view of another embodiment of the shoulder dressing of FIG. 8, according to an exemplary embodiment.

FIG. 11 is a top view of yet another embodiment of the shoulder dressing of FIG. 8, according to an exemplary embodiment.

FIG. 12 is an illustration of the shoulder dressing of FIG. 8 applied to a patient in a first application location and orientation, according to an exemplary embodiment.

FIG. 13 is an illustration of the shoulder dressing of FIG. 8 applied to a patient in a second application location and orientation, according to an exemplary embodiment.

FIG. 14 is an illustration of the shoulder dressing of FIG. 8 applied to a patient in a third application location and orientation, according to an exemplary embodiment.

### DETAILED DESCRIPTION

### Overview

Referring generally to the FIGURES, a wound therapy system for treating wounds of curved body parts is shown, according to various embodiments. More specifically, the wound therapy system is for treating wounds in the shoulder area. The wound therapy system includes a wound dressing and a negative pressure wound therapy (NPWT) system. The phrase “negative pressure” means a pressure less than an ambient or atmospheric pressure. While the amount and nature of reduced pressure applied to the wound treatment area can vary according to the application, the reduced pressure typically is between −5 mm Hg and −500 mm Hg and more typically between −100 mm Hg and −300 mm Hg.

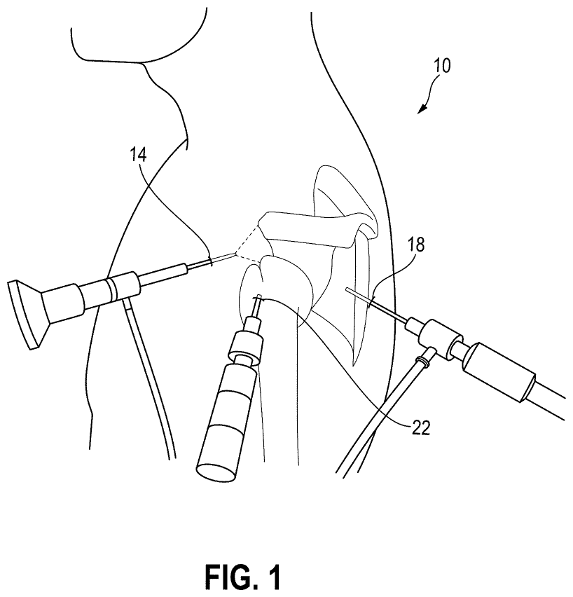

FIG. 1 illustrates an exemplary shoulder 10 of a patient undergoing arthroscopic shoulder surgery. Arthroscopic shoulder surgeries typically include a first incision 14 at front of an upper portion of a patient's shoulder, second incision 18 at a back of an upper portion of a patient's shoulder, and a third incision 22 at an upper portion of a patient's arm. The wound treatment area includes the first incision 14, healthy tissue surrounding the first incision 14, the second incision 18, healthy tissue surrounding the second incision 18, the third incision 22, and healthy tissue surrounding the second incision 18. The wound dressings described herein are configured to substantially cover the wound treatment area and apply NPWT to the wound treatment area. During arthroscopic shoulder surgery, several liters of fluid are pumped into the shoulder area. Applying NPWT to the entire wound treatment area can facilitate patient healing by lifting the healthy tissue surrounding the incisions, which facilitates absorption of the fluid by the lymphatic system of the patient's body. In some embodiments, the NPWT system can remove excess fluid that cannot be absorbed by the body. For example, fluid (wound exudate, fluid injected during surgery, etc.) can drain from the wound treatment area via the negative pressure conduit or a dedicated drain line. Fluid can be collected by a removed fluid reservoir of the NPWT system.

In some embodiments, the wound dressing is substantially T-shaped and includes a first lobe, a second lobe, and a third lobe. The first lobe and the second lobe are generally aligned and the third lobe is generally perpendicular to the first lobe and the second lobe. The first lobe and the second lobe are substantially half-ellipses and the third lobe is substantially circular. The first lobe and the second lobe are connected to the third lobe by a connection portion that is narrower than the first lobe, the second lobe, and the third lobe. The wound dressing is shaped to wrap around the shoulder of the patient. The first lobe is configured to overlie the first incision 14 and the healthy tissue surrounding the first incision 14 at the front portion of the patient's shoulder. The second lobe is configured to overlie the second incision 18 and the healthy tissue surrounding the second incision 18 at the back portion of the patient's shoulder. The third lobe is configured to overlie the third incision 22 and the healthy tissue surrounding the third incision 22 at the upper portion of the patient's arm. In some embodiments, the wound dressing includes a concave contour that is generally aligned with the third lobe. The concave contour is configured to prevent the wound dressing from overlying the patient's trapezius muscle. The shape of the wound dressing is generally symmetric to allow placement of the wound dressing on either the left or the right shoulder.

In some embodiments, the wound dressing has a substantially 3D-shape configured to conform to the shoulder wound treatment area. In such an embodiment, the wound dressing forms an elbow-shaped channel that includes a first portion and a second portion that is angled relative to the first portion. In some embodiments, the second portion is at a substantially obtuse angle relative to the first portion. The first portion is configured to overlie a first incision and the surrounding healthy tissue at a front portion of the patient's shoulder, a top portion of the patient's shoulder, and a second incision and the surrounding healthy tissue at a back portion of the patient's shoulder. In some embodiments, a first lobe and a second lobe extend from the first portion. In some embodiments, the first lobe and the second lobe are generally perpendicular to the first portion. The first lobe is configured to overlie the first incision and healthy tissue surrounding the first incision. The second lobe is configured to overlie the second incision and healthy tissue surrounding the first incision.

The wound dressing can be used in conjunction with an immobilization device such as a sling or a belt that is configured to immobilize a patient's arm relative to the patient's torso to immobilize the patient's shoulder joint. A negative pressure source or pump and a removed fluid container are integrated into the immobilization device. The wound dressing includes a negative pressure interface that facilitates fluid communication between the wound dressing and a negative pressure conduit that is coupled the negative pressure source. A portion of the negative pressure conduit proximate the NPWT system is positioned within the immobilization device. Integration of the NPWT system with the immobilization device allows the patient to conveniently transport the negative pressure source while the patient is undergoing NPWT.

In some embodiments, the wound dressing is configured to drain excess fluid from the wound treatment area. In such embodiments, the removed-fluid container can be configured to store a fluid removed from the wound treatment area (e.g., wound exudate, fluid injected during surgery, etc.). In some embodiments, the removed fluid container is positioned upstream of the negative pressure source so that fluid can drain from the wound dressing via the negative pressure conduit and accumulate in the removed-fluid container. In other embodiments, the removed-fluid container can be fluidly coupled to the wound treatment area via a fluid removal line that is separate from the negative pressure conduit. The NPWT can help reduce the chance of the wounds developing seroma, scaring, infection, or other adverse complications.

Additional features and advantages of the wound therapy system are described in detail below.

### Wound Dressing

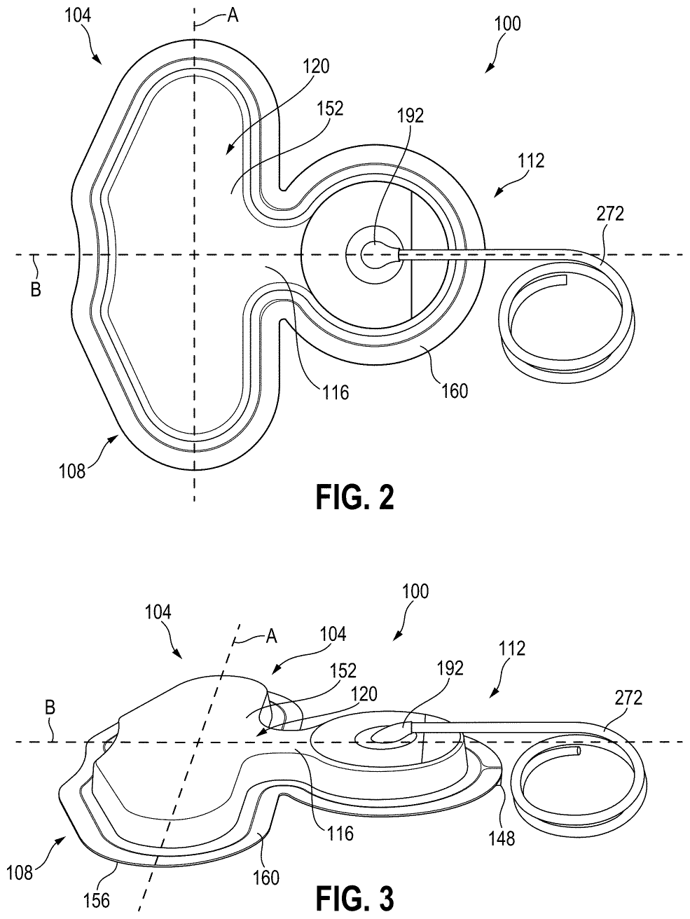

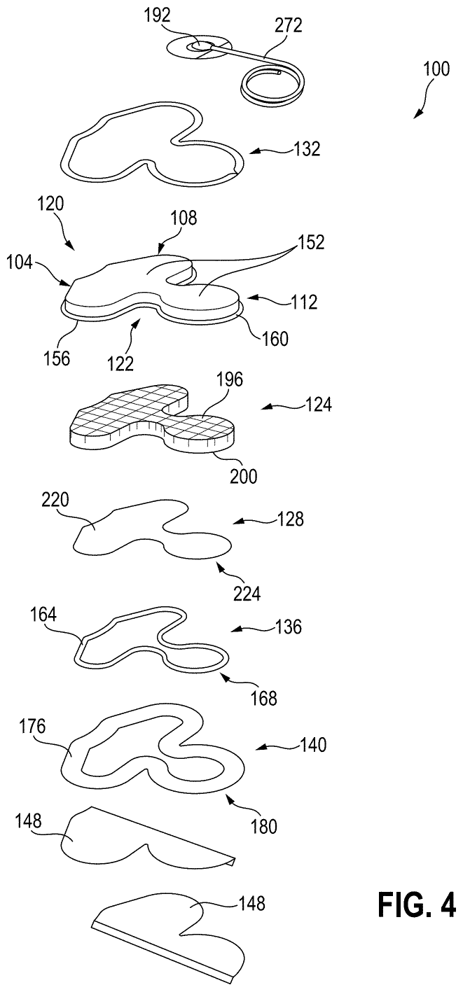

Referring now to FIGS. 2-4, a wound dressing 100 is shown, according to an exemplary embodiment. FIG. 2 is a front view of the wound dressing 100. FIG. 3 is a perspective view of the wound dressing 100. FIG. 4 is an exploded view illustrating several layers 120-148 of the wound dressing 100.

In various embodiments, the wound dressing 100 can be formed as a substantially flat sheet for topical application to wounds. The wound dressing 100 is generally planar, but can wrap around a shoulder of a patient to conform to the three-dimensional shape of a wound treatment area at the shoulder of the patient. The wound dressing 100 is substantially T-shaped and includes a first lobe 104, a second lobe 108, and a third lobe 112. The first lobe 104 and the second lobe 108 are substantially half-ellipses and are aligned along an axis A. The third lobe 112 is substantially circular and is connected to the first lobe 104 and the second lobe 108 by a connection portion 116 that is narrower than the third lobe 112. The third lobe 112 is substantially perpendicular to the first lobe 104 and the second lobe 108. The first lobe 104 is configured to overlie an incision at a front portion of a patient's shoulder and healthy tissue surrounding the incision. The second lobe 108 is configured to overlie an incision at a back portion of a patient's shoulder 10 and healthy tissue surrounding the incision. The third lobe 112 is configured to overlie an incision at an upper portion of a patient's arm and healthy tissue surrounding the incision. The wound dressing 100 is substantially symmetric about an axis B so that the wound dressing 100 can be deployed on a patient's right shoulder or a patient's left shoulder without requiring modification.

The wound dressing 100 is shown to include a plurality of layers, including a drape layer 120, a manifold layer 124, a wound-interface layer 128, a semi-rigid support layer 132, a first adhesive layer 136, and a second adhesive layer 140. In some embodiments, the wound dressing 100 includes a removable cover sheet 148 to cover the manifold layer 124, the wound-interface layer 128, and the second adhesive layer 140 before use.

### Drape Layer

The drape layer 120 is shown to include a first surface 152 and a second, wound-facing, surface 156 opposite the first surface 152. When the wound dressing 100 is applied to a wound, the first surface 152 faces away from the wound, whereas the second surface 156 faces toward the wound. The drape layer 120 supports the manifold layer 124 and the wound-interface layer 128 and provides a barrier to passage of microorganisms through the wound dressing 100. The drape layer 120 is configured to provide a sealed space over a wound or incision. In some embodiments, the drape layer 120 is an elastomeric material or may be any material that provides a fluid seal. “Fluid seal” means a seal adequate to hold pressure at a desired site given the particular reduced-pressure subsystem involved. The term “elastomeric” means having the properties of an elastomer and generally refers to a polymeric material that has rubber-like properties. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. As non-limiting examples, the drape layer 120 may be formed from materials that include a silicone, 3M Tegaderm® drape material, acrylic drape material such as one available from Avery, or an incise drape material.

The drape layer 120 may be substantially impermeable to liquid and substantially permeable to water vapor. In other words, the drape layer 120 may be permeable to water vapor, but not permeable to liquid water or wound exudate. This increases the total fluid handling capacity (TFHC) of wound dressing 100 while promoting a moist wound environment. In some embodiments, the drape layer 120 is also impermeable to bacteria and other microorganisms. In some embodiments, the drape layer 120 is configured to wick moisture from the manifold layer 124 and distribute the moisture across the first surface 152.

As shown in FIG. 4, the drape layer 120 defines a cavity 122 for receiving the manifold layer 124, the wound-interface layer 128, and the first adhesive layer 136. The manifold layer 124, the wound-interface layer 128, and the first adhesive layer 136 can have a similar perimeter or profile. In some embodiments, a perimeter of the drape layer 120 extends beyond (e.g. circumscribes) the perimeter of the manifold layer 124 to provide a margin 160. The first adhesive layer 136 includes a first surface 164 and a second, wound-facing surface 168. Both first surface 164 and the second surface 168 are coated with an adhesive, such as an acrylic adhesive, a silicone adhesive, and/or other adhesives. The first surface 164 of the first adhesive layer 136 is secured to the second surface 172 of the wound-interface layer 128. The second surface 168 of the first adhesive layer 136 is secured to the second adhesive layer 140. The second adhesive layer 140 includes a first surface 176 and a second, wound-facing surface 180. The second surface 168 of the first adhesive layer 136 is secured to the first surface 176 of the second adhesive layer 140. The second surface 180 of the second adhesive layer 140 is coated with an acrylic adhesive, a silicone adhesive, and/or other adhesives. The adhesive applied to the second surface 180 of the second adhesive layer 140 is intended to ensure that the wound dressing 100 adheres to the surface of the patient's tissue and that the wound dressing 100 remains in place throughout the wear time. The second adhesive layer 140 has a perimeter or profile that is similar to a perimeter or profile of the margin 160. In the illustrated embodiment, the first surface 176 of the second adhesive layer 140 is welded to the margin 160. In other embodiments, the first surface 176 of the second adhesive layer is secured to the margin 160 using an adhesive, such as an acrylic adhesive, a silicone adhesive, or another type of adhesive. The margin 160 and/or the second adhesive layer 140 may extend around all sides of the manifold layer 124 such that the wound dressing 100 is a so-called island dressing. In other embodiments, the margin 160 and/or the second adhesive layer 140 can be eliminated and the wound dressing 100 can be adhered to the patient's tissue using other techniques. In some embodiments, the first adhesive layer 136, and the second adhesive layer 140 can collectively form a base layer that includes an adhesive on both sides that is (i) configured to secure the drape layer 120 to the manifold layer 124, the optional wound-interface layer 128, and (ii) configured to secure the wound dressing 100 to a patient's tissue. In some embodiments, the base layer can be integrally formed with the drape layer 120. In some embodiments, the base layer can be a layer of a polyurethane film having a first surface and a second, wound-facing surface. Both the first surface and the second surface can be coated with an adhesive (such as an acrylic or silicone adhesive). In some embodiments, the wound-facing surface of the base layer can include a hydrocolloid adhesive.

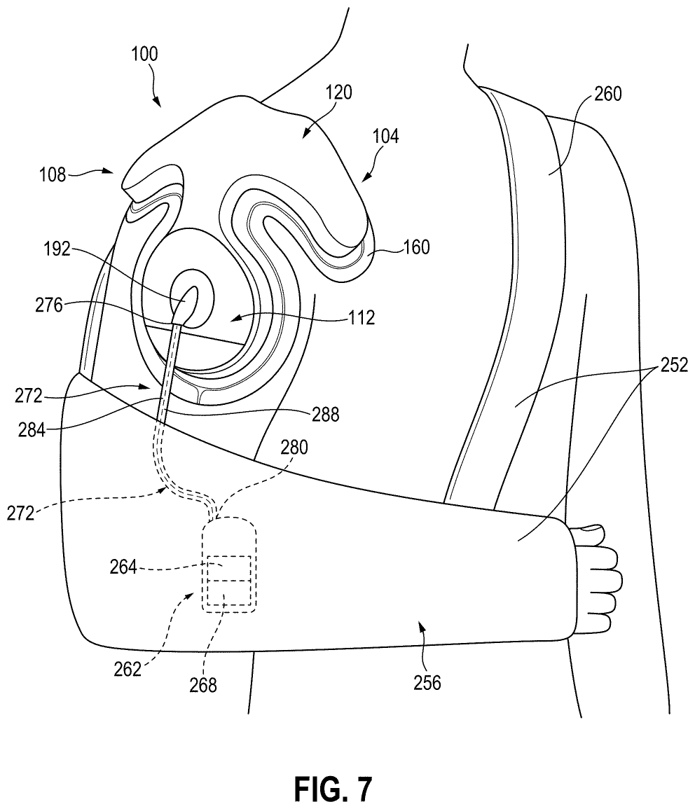

In some embodiments, a reduced-pressure interface 192 can be integrated with the drape layer 120. The reduced-pressure interface 192 can be in fluid communication with the negative pressure system through a negative pressure conduit 272. The reduced-pressure interface 192 is configured to allow fluid communication between a negative pressure source 268 (FIG. 7) and the wound dressing 100 (e.g., through the drape layer 120) via the negative pressure conduit 272 coupled between the reduced-pressure interface 192 and the negative pressure source 268 such that negative pressure generated by the negative pressure source 268 can be applied to the wound dressing 100 (e.g., through the drape layer 120). In some embodiments, the reduced-pressure interface 192 can be integrated (e.g., integrally formed) with the drape layer 120. In other embodiments, the reduced-pressure interface 192 can be separate from the drape layer 120 and configured to be coupled to the drape layer 120 by a user. In the illustrated embodiment, the reduced-pressure interface 192 is positioned above the third node 112. In other embodiments, the reduced-pressure interface 192 can be positioned elsewhere on the drape layer 120.

With continued reference to FIG. 4, the semi-rigid support layer 132 is positioned above the first surface 152 of the drape layer 120. The semi-rigid support layer 132 is spaced from but proximate the margin 160 and the second adhesive layer 140. The semi-rigid support layer 132 is made of a semi-rigid material and helps the wound dressing 100 maintain rigidity before the wound dressing 100 is secured to the surface of the patient. The semi-rigid support layer 132 is intended to be removed from the drape layer 120 after the wound dressing 100 has been secured to the patient's tissue.

In some embodiments, the second surface 156 of the drape layer 120 contacts the manifold layer 124. The second surface 156 of the drape layer 120 may be adhered to the manifold layer 124 or may simply contact the manifold layer 124 without the use of an adhesive.

In some embodiments, the adhesive applied to the second surface 156 of the drape layer 120 is moisture vapor transmitting and/or patterned to allow passage of water vapor therethrough. The adhesive may include a continuous moisture vapor transmitting, pressure-sensitive adhesive layer of the type conventionally used for island-type wound dressings (e.g. a polyurethane-based pressure sensitive adhesive).

### Manifold Layer

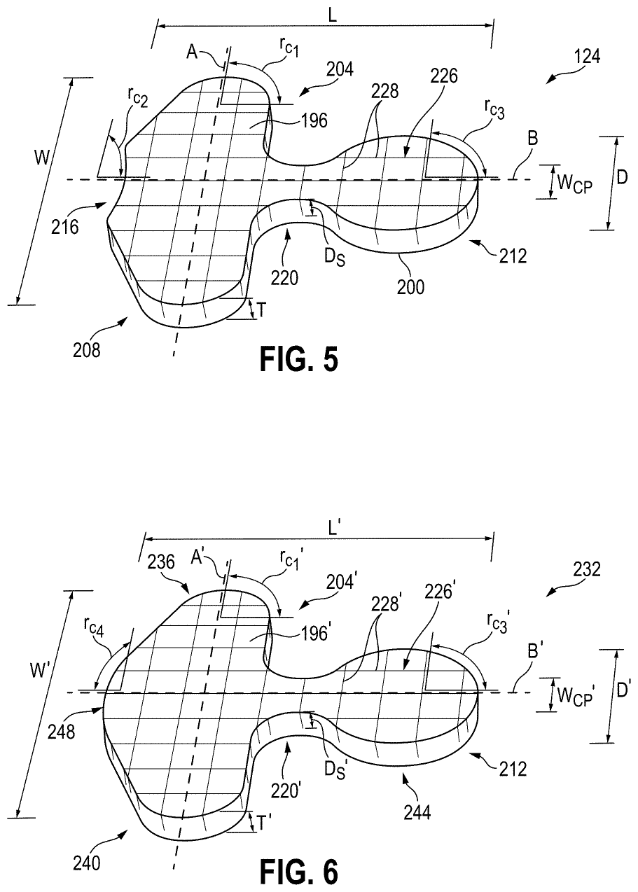

Referring to FIG. 5, the manifold layer 124 is shown to include a first surface 196 and a second, wound-facing surface 200 opposite the first surface 196. When the wound dressing 100 is applied to a wound, the first surface 196 faces away from the wound, whereas the second surface 200 faces toward the wound. In some embodiments, the first surface 196 of the manifold layer 124 contacts the second surface 156 of the drape layer 120. In some embodiments, the second surface 200 of the manifold layer 124 contacts the wound-interface layer 128. The manifold layer 124 is configured for transmission of negative pressure to the patient's tissue at and/or proximate a wound and/or incision. The manifold layer 124 is configured to wick fluid (e.g. exudate) from the wound and includes in-molded manifold layer structures for distributing negative pressure throughout the wound dressing 100 during negative pressure wound therapy treatments.

The manifold layer 124 can be made from a porous and permeable foam-like material and, more particularly, a reticulated, open-cell polyurethane or polyether foam that allows good permeability of wound fluids while under a reduced pressure. One such foam material that has been used is the V.A.C.® Granufoam™ material that is available from Kinetic Concepts, Inc. (KCl) of San Antonio, Tex. Any material or combination of materials might be used for the manifold layer 124 provided that the manifold layer 124 is operable to distribute the reduced pressure and provide a distributed compressive force along the wound treatment area.

The reticulated pores of the Granufoam™ material that are in the range from about 400 to 600 microns, are preferred, but other materials may be used. The density of the manifold layer material, e.g., Granufoam™ material, is typically in the range of about 1.3 lb/ft³-1.6 lb/ft³ (20.8 kg/m³-25.6 kg/m³). A material with a higher density (smaller pore size) than Granufoam™ material may be desirable in some situations. For example, the Granufoam™ material or similar material with a density greater than 1.6 lb/ft³ (25.6 kg/m³) may be used. As another example, the Granufoam™ material or similar material with a density greater than 2.0 lb/ft³ (32 kg/m³) or 5.0 lb/ft³ (80.1 kg/m³) or even more may be used. The more dense the material is, the higher compressive force that may be generated for a given reduced pressure. If a foam with a density less than the tissue at the tissue site is used as the manifold layer material, a lifting force may be developed. In one illustrative embodiment, a portion, e.g., the edges, of the wound dressing 100 may exert a compressive force while another portion, e.g., a central portion, may provide a lifting force.

The manifold layer material may be a reticulated foam that is later felted to thickness of about one third (¼) of the foam's original thickness. Among the many possible manifold layer materials, the following may be used: Granufoam™ material or a Foamex® technical foam (www.foamex.com). In some instances, it may be desirable to add ionic silver to the foam in a microbonding process or to add other substances to the manifold layer material such as antimicrobial agents. The manifold layer material may be isotropic or anisotropic depending on the exact orientation of the compressive forces that are desired during the application of reduced pressure. The manifold layer material may also be a bio-absorbable material.

As shown in FIGS. 2-5, the manifold layer 124 is generally symmetrical about the axis B. The manifold layer 124 is substantially T-shaped and includes a first lobe 204, a second lobe 208, and a third lobe 212. The manifold layer 124 can have a length L ranging from approximately 7.44 inches to 11.16 inches. In some embodiments, the length L is approximately 9.3 inches. The manifold layer 124 can have a width W ranging from approximately 7.76 inches to approximately 11.64 inches. In some embodiments, the width W is approximately 9.7 inches. The manifold layer 124 can have a thickness T ranging from approximately 0.64 inches to 0.96 inches. In some embodiments, the thickness T is approximately 0.8 inches.

The first lobe 204 and the second lobe 208 are substantially elliptical. The first lobe 204 and the second lobe 208 can each have a radius of curvature rc₁ ranging from approximately 1.04 inches to 1.56 inches. In some embodiments, the first lobe 204 and the second lobe 208 can each have a radius of curvature of approximately 1.3 inches. The first lobe 204 and the second lobe are substantially aligned along the axis A. A concave portion 216 extends along a portion of the perimeter of the wound dressing 100 that is between the first lobe 204 and the second lobe 208. The concave portion 216 is substantially aligned with the third lobe 212 along the axis B. The concave portion 216 is positioned to prevent the wound dressing from overlying a trapezius muscle of a patient and/or contacting the patient's neck when the wound dressing 100 is secured to the patient's shoulder. The concave portion 216 can have a radius of curvature rc₂ ranging from approximately 4 inches to approximately 6 inches. In some embodiments, the radius of curvature rc₂ can be approximately 5 inches.

The third lobe 212 is substantially perpendicular to the first lobe 204 and the second lobe 208. The third lobe 212 is subsantially circular. The third lobe can have a radius of curvature rc₃ ranging from approximately 1.6 inches to approximately 2.4 inches. In some embodiments, the radius of curvature rc₃ can be approximately 2.0 inches. The third lobe 212 is connected to the first lobe 204 and the second lobe 208 by the connecting portion 220. The connecting portion 220 has a width W_{CP} smaller than a diameter D of the third lobe 212. The width W_{CP} of the connecting portion can range from approximately 1.2 inches to approximately 1.8 inches. In some embodiments, the width W_{CP} can be approximately 1.5 inches.

As is best shown in FIG. 5, a scoring pattern 226 is formed in the first surface 196 of the manifold layer 124. The scoring pattern 226 is shown for example as an arrangement of “slits” or scores (e.g., “mango-cuts”) formed in the manifold layer 124 (e.g. formed by laser-scoring or other suitable processes). More particularly, the scoring pattern 226 is cut into the first surface 196 of the manifold layer 124. In the embodiment of FIG. 5, the scoring pattern 226 extends between the first surface 196 and the second surface 200 but does not extend completely to the second surface 200. The scoring pattern 226 can have a depth Ds that can range from approximately 0.2 inches to 0.5 inches. In some embodiments, the depth Ds is approximately 0.28 inches. According to the illustrated embodiment, the scoring pattern 226 is a generally square pattern. However, in other embodiments, the scoring pattern 226 can be a different geometrical pattern. When the wound dressing 100 is used on a generally flat (e.g. two-dimensional) portion of the wound treatment area, such as for example a front of a shoulder or a back of a patient's shoulder, the scores 228 of the scoring pattern 226 are generally vertical and are in close proximity to adjacent scores 228 of the scoring pattern 226. In instances when the wound dressing 100 is secured to a curved (e.g. three-dimensional) surface, such as a transition portion of the surface that extends between the front of the shoulder and the top of the shoulder, the back of the shoulder and the top of the shoulder, and the shoulder and the top of the arm, the scores 228 of the scoring pattern 226 splay apart to facilitate bending of the manifold layer 124 so that the manifold layer 124 closely conforms to the shape of the wound treatment area. The scoring pattern 226 allows the manifold layer 124 to conform to both substantially flat surfaces and curved surfaces at the wound treatment area.

FIG. 6 illustrates a manifold layer 232 according to another embodiment. The manifold layer 232 is generally similar to the manifold layer 124. The manifold layer 232 can be incorporated into the wound dressing 100 as described above with respect to the manifold layer 124. Like numbers are indicated by the same number and parts of the manifold layer 232 are indicated using the prime symbol “′”.

As shown in FIGS. 2-5, the manifold layer 232 is generally symmetrical about the axis B. The manifold layer 232 is substantially T-shaped and includes a first lobe 236, a second lobe 240, and a third lobe 244. The manifold layer 232 can have a length L′ ranging from approximately 7.44 inches to 11.16 inches. In some embodiments, the length L′ is approximately 9.3 inches. The manifold layer 232 can have a width W′ ranging from approximately 7.76 inches to approximately 11.64 inches. In some embodiments, the width W′ is approximately 9.7 inches. The manifold layer 232 can have a thickness T′ ranging from approximately 0.64 inches to 0.96 inches. In some embodiments, the thickness T′ is approximately 0.8 inches.

The manifold layer 232 includes the first lobe 236, the second lobe 240, and the third lobe 244 described above with respect to FIG. 5. The first lobe 236 and the second lobe 240 are substantially elliptical. The first lobe 236 and the second lobe are substantially aligned along the axis A′. A convex portion 248 extends along a portion of the perimeter of the wound dressing 100 that is between the first lobe 236 and the second lobe 240. The convex portion 248 is substantially aligned with the third lobe 244 along the axis B′. The convex portion 248 can have a radius of curvature rc₄ ranging from approximately 4 inches to approximately 6 inches. In some embodiments, the radius of curvature rc₄ can be approximately 5 inches.

### Wound-Interface Layer

The wound-interface layer 128 is shown to include a first surface 222 and a second, wound-facing surface 224 opposite the first surface 222. When the wound dressing 100 is applied to the wound, the first surface 222 faces away from the wound, whereas the second surface 224 faces toward the wound. In some embodiments, the first surface 222 of the wound-interface layer 128 contacts the second surface 224 of the manifold layer 124. In some embodiments, the second surface 224 of the wound-interface layer 128 contacts the patient's tissue. In some embodiments, the wound dressing 100 may not include the wound-interface layer 128.

The wound-interface layer 128 is made of a wicking material that is fluid-permeable and intended to not irritate the patient's tissue. In the illustrated embodiment, the wound-interface layer is a polyester pique-knit fabric, such as Milliken Fabric. In other embodiments, other permeable and non-irritating fabrics can be used. The wound-interface layer 128 can also be treated with antimicrobial materials. In the illustrated embodiment, the wound-interface layer 128 includes silver ions as an antimicrobial material. Other antimicrobial materials may be used in other embodiments.

### Integrated Immobilization Device and NPWT System

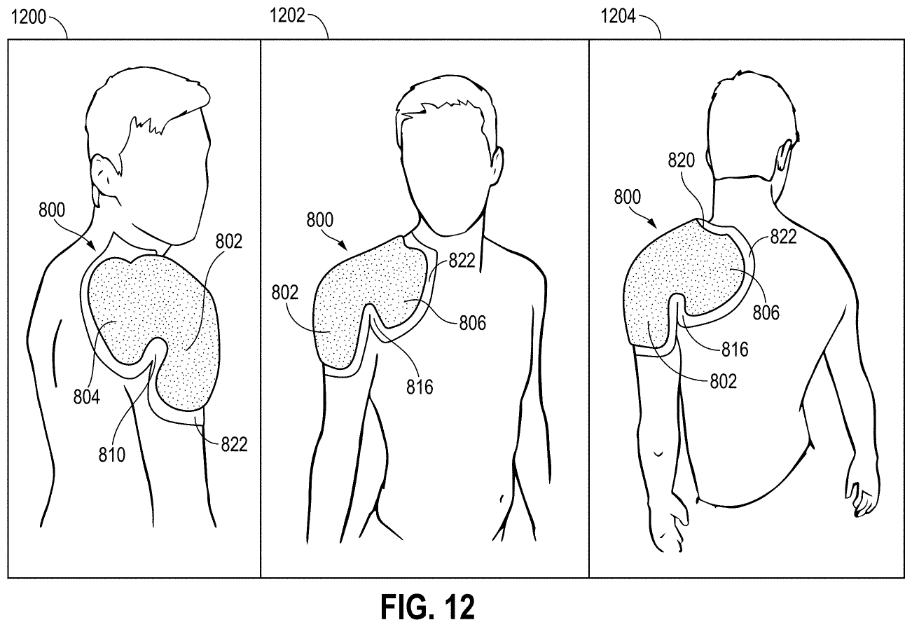

Referring now to FIG. 7, the wound dressing 100 is used in conjunction with an immobilization device 252. The immobilization device 252 is configured to immobilize a patient's arm relative to the patient's shoulder to restrict movement of the patient's shoulder. In the embodiment illustrated in FIG. 7, the immobilization device 252 is a sling. The sling includes an arm-receiving portion 256 and a shoulder strap 260. The arm-receiving portion 256 is configured to receive at least the forearm and elbow of the arm corresponding to the injured shoulder. The shoulder strap 260 is coupled to the arm-receiving portion 256 and is configured to be positioned over a patient's uninjured shoulder to support the arm corresponding to the patient's injured shoulder. In some embodiments, the sling includes a belt (not shown) configured to immobilize the arm-receiving portion 256 of the sling relative to the patient's torso. In other embodiments, the immobilization device 252 can be a belt as illustrated below in FIG. 12.

The NPWT system 262 further includes a removed fluid container 264 and a negative pressure source or pump 268 that are in fluid communication with the wound dressing 100 via the negative pressure conduit 272. In some embodiments, the pump 268 can be a powered pump 268. In such an embodiment, the NPWT system 262 further includes a battery configured to power the pump 268. In other embodiments, the pump 268 is an unpowered pump. In such an embodiment, the pump 268 can be hand-actuated by the patient. The removed fluid container 264 can be configured to store a fluid removed from the incisions 14, 18, 22 (FIG. 1). Removed fluid can include, for example, wound exudate (e.g., bodily fluids), air, fluid that was injected into the wound treatment area during surgery, or any other type of fluid which can be removed from the incision 240 during wound treatment.

The NPWT system 262 is coupled to the wound dressing 100 by the negative pressure conduit 272. The negative pressure conduit 272 has a first end 276 coupled to the reduced-pressure interface 192 of the wound dressing 100 and a second end 280 coupled to the NPWT system 262. In the illustrated embodiment, the negative pressure conduit 272 is a multi-lumen conduit. The negative pressure conduit 272 includes a first lumen 284 and a second lumen 288. The first lumen 284 is configured to apply negative pressure to the wound dressing 100 and to draw exudate into the removed fluid container 264. The second lumen 288 is configured for sensing the pressure of the wound dressing 100. One such NPWT system 262 including a multi-lumen conduit is the SensaT.R.A.C.™ system that is available from Kinetic Concepts, Inc. (KCl) of San Antonio, Tex.

Returning to FIG. 7, the NPWT system 262 is integrated with the immobilization device 252. As illustrated in FIG. 7, the NPWT system 262 is secured within the arm-receiving portion 256 of the immobilization device 252. For example, the NPWT system 262 can be positioned within a pocket of the arm-receiving portion 256, sewn into the arm-receiving portion 256, secured within the arm-receiving portion 256 of the immobilization device 252 using a detachable adhesive such as Velcro, etc. As illustrated in FIG. 7, a portion of the negative pressure conduit 272 proximate the NPWT system 262 is integrated with the arm-receiving portion 256. For example, the negative pressure conduit 272 can be positioned within a passageway of the arm-receiving portion 256, secured within the arm-receiving portion 256 of the immobilization device 252 using a detachable adhesive such as Velcro, etc. Integration of the NPWT system 262 within the immobilization device 252 allows the patient to conveniently transport the NPWT system 262 while the patient is undergoing NPWT.

### Deployment of the Dressing

FIG. 7 illustrates the wound dressing 100 deployed at a representative illustration of a patient's torso. The patient's arm proximate the wounded shoulder is immobilized relative to the patient's torso by the immobilization device 252 to immobilize the shoulder joint. While wound dressing 100 is shown in FIG. 7, the manifold layer 232 can be deployed in a similar manner Referring briefly to FIG. 1, the wound treatment area includes the first incision 14 and surrounding healthy tissue at a front of the patient's shoulder, the second incision 18 and surrounding healthy tissue at a back of the patient's shoulder, and the third incision 22 and surrounding healthy tissue at a top of the patient's arm. As illustrated in FIG. 7, the wound dressing 100 does not over the patient's armpit. As is apparent from comparison of FIGS. 1 and 7, the wound dressing 100 is sized to cover the surface including the entire wound treatment area. A further advantage of covering the entire wound area is that the wound dressing 100 can provide NPWT to the whole wound treatment area to generate negative pressure and lifting forces over the wound treatment area to facilitate wound healing and to facilitate absorption of the fluid injected during surgery by the lymphatic system. In some embodiments, the wound dressing 100 can be used with topically applied pharmaceutical compounds. For example, the wound dressing 100 can be used in conjunction with a silicone gel applied proximate the first incision 14, the second incision 18, and the third incision 22. The silicone gel can reduce scarring at or near the incisions 14, 18, 22.

As illustrated in FIG. 7, the reduced-pressure interface 192 is positioned over the third lobe 212. In other embodiments, the reduced-pressure interface 192 can be positioned elsewhere on the drape layer 120 of the wound dressing 100. The negative pressure conduit 272 extends from the reduced-pressure interface 192 and extends along the patient's arm and into the immobilization device 252. As illustrated using phantom lines, the negative pressure conduit 272 is coupled to the NPWT system 262 integrated with the immobilization device 252. Due to the symmetric shape of the wound dressing 100, the wound dressing can be used to treat wounds in both the left shoulder and the right shoulder.

To deploy the wound dressing 100 to treat a wound treatment area at a shoulder of the patient, a healthcare practitioner removes the cover sheet 148 from the wound dressing 100. The healthcare practitioner then orients the wound dressing 100 relative to the patient's shoulder such that the first lobe 104 overlies an incision and surrounding healthy tissue at a front of a patient's shoulder and the second lobe 108 overlies an incision and healthy tissue at a back of the patient's shoulder. The healthcare practitioner then orients the wound dressing 100 such that the third lobe 112 overlies an incision and surrounding healthy tissue at an upper portion the patient's arm proximate the wounded shoulder. The healthcare practitioner then applies pressure around the perimeter of the margin 160 of the drape layer 120 to secure the second adhesive layer 140 to the patient's tissue. The healthcare practitioner then immobilizes the patient's arm relative to the patient's torso using the immobilization device 252. The healthcare practitioner then inserts the negative pressure conduit 272 into the immobilization device 252 and couples the negative pressure conduit 272 to the NPWT system 262. The healthcare practitioner then actuates the NPWT system 262 to apply negative pressure to the wound treatment area.

### Joint Dressing

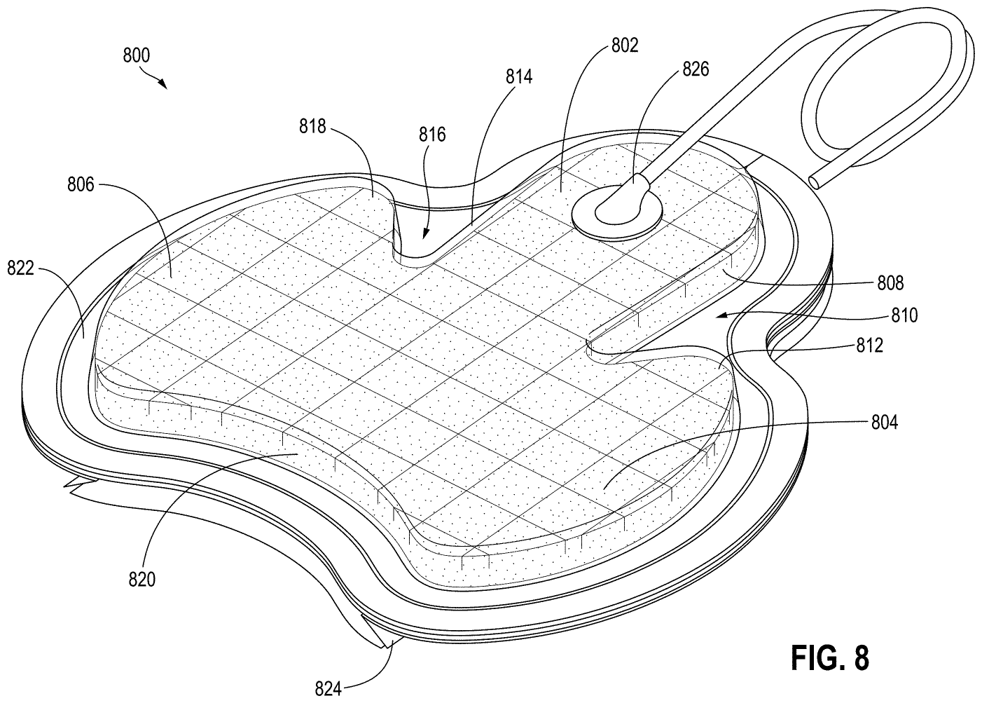

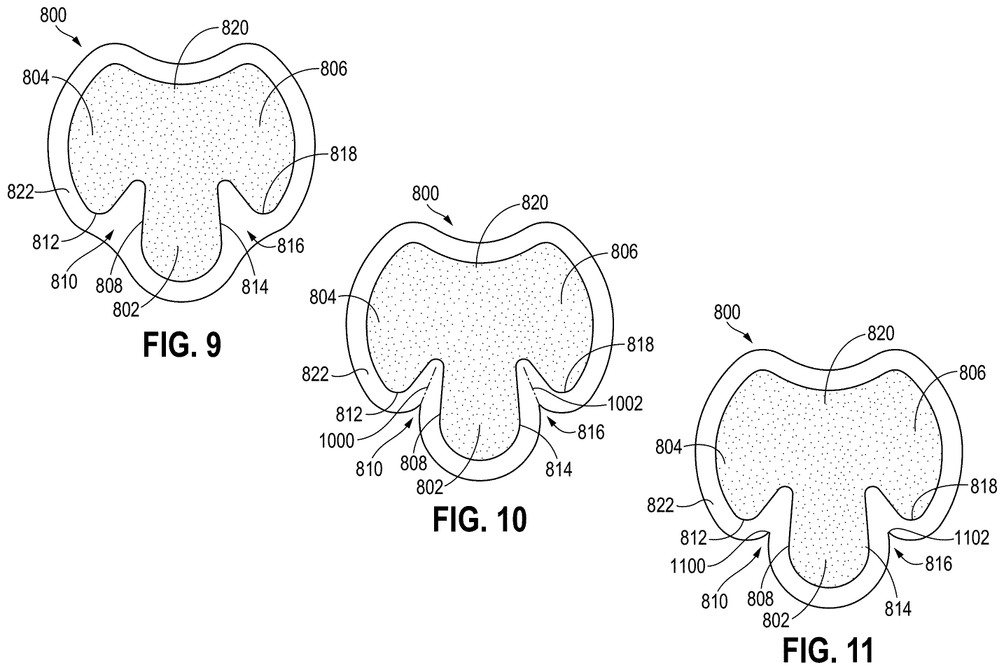

Referring generally to FIGS. 8-14, another dressing for use with negative pressure therapy is shown, according to various example embodiments. In particular, a perspective view of a dressing 800 is shown in FIG. 8, and top views of various embodiments of the dressing 800 are shown in FIGS. 9-11. FIGS. 12-14 show example applications of the dressing 800 to a patient. These illustrations are described in detail below. The dressing 800 can be included in a negative pressure therapy system, for example included in a kit that also includes a negative pressure source (e.g., pump), tubing or other fluid conduit, and/or a shoulder immobilization device (e.g., sling).

The dressing 800 is shown for illustration purposes as configured to be placed at a shoulder region of a patient in multiple orientations as desired by a clinician and to provide coverage of a large extent of the shoulder of the patient. The dressing 800 is configured for use with a negative pressure source to allow a negative pressure to be established and maintained at the shoulder of the patient. The dressing 800 can be used to promote healing of wounds at the shoulder (e.g., surgical incisions) and/or to help reduce swelling at the shoulder of the patient. The dressing 800 can also be applied to and positioned at various other anatomical regions, for example a neck and upper back region, or a joint region such as a shoulder, elbow, hip, knee, or ankle joint, or other region with suitable geometry.

The dressing 800 can be made up of the multiple layers and materials described above for other dressing embodiments, for example as shown in FIGS. 4 and 6 and described with reference thereto above. For example, a manifold layer of the dressing 800 can be positioned between a drape layer and a patient interface layer, and may be scored to facilitate bending and conformance of the dressing 800 as described in detail above. The dressing 800 is configured in a similar manner as the embodiments described above with further features emphasized in the following description.

The dressing 800 includes a body portion 802, a first wing 804 extending from the body portion 802 in a first lateral direction, and a second wing 806 extending from the body portion 802 in a second lateral direction opposite the first lateral direction. The body portion 802, the first wing 804, and the second wing 806 may be made up of and define a drape layer, a manifold layer, and a wound interface layer. In other words, the drape layer, the manifold layer, and the wound interface layer are all shaped into the body portion 802, first wing 804, and second wing 806 and layered to form the dressing 800. The terms “first” and “second” in this description are used as labels to differentiate between different portions of the dressing 800 and are not intended to imply a hierarchy.

The body portion 802 extends in a longitudinal direction, with the first wing 804 and the second wing 806 extending from the body portion 802 at an area spaced apart from one end of the body portion 802. The first wing 804 has an elliptical shape and extends both away from a first side 808 of the body portion 802 and along a longitudinal direction of the body portion 802, such that a gap 810 is provided between the first side 808 of the body portion 802 and a first tip 812 of the first wing 804. The body portion 802 extends along the longitudinal direction beyond (further than) the first tip 812. The gap 810 may be defined by an angle between the first side 808 of the body portion 802 and the first wing 804 in a range between fifteen and forty-five degrees, for example approximately thirty degrees.

The second wing 806 has an elliptical shape and extends both away from a second side 814 of the body portion and along the longitudinal direction of the body portion 802, such that a gap 816 is provided between the second side 814 of the body portion 802 and a second tip 818 of the second wing 806. The gap 816 may be defined by an angle between the second side 814 and the second wing 806 in a range between fifteen and forty-five degrees, for example approximately thirty degrees.

In the embodiments shown, the dressing 800 is symmetric across a longitudinal axis of the body portion 802, with the second wing 806 shaped substantially as a mirror-image of the first wing 804 with substantially the same shape and size (e.g. in the shape of a “battle axe” according to one embodiment). In other embodiments, the first wing 804 and the second wing 806 may have different dimensions or shapes from one another.

As shown, the body portion 802 includes a concave edge 820 that extends between the first wing 804 and the second wing 806 along an end of the body portion 802. The concave edge 820 is configured to facilitate conformance of the dressing 800 to a shoulder region of a patient, for example by providing space for a trapezius muscle or neck of the patient. Accordingly, the concave edge 820 can help the dressing 800 conform to the patient by extending around rather than over a trapezius muscle or other anatomical feature that protrudes normal to a surface of surrounding tissue.

The dressing 800 is shown to include an adhesive border 822. The adhesive border 822 extends around a periphery of the dressing 800 and is configured to adhere the dressing 800 to skin of a patient. The adhesive border 822 can provide a substantially air-tight seal between the dressing 800 and the patient's skin. The adhesive border 822 may also be configured to adhere to itself or other portions of the dressing 800 in various applications. The adhesive border 822 is thereby configured for use in securing the dressing 800 in position on the patient (e.g., at the patient's shoulder) and for providing a sealed volume between the dressing 800 and the patient's skin.

From the perspective view of FIG. 8, a removable backing 824 is shown coupled to the patient-facing side of the dressing 800. The removable backing 824 protects the patient-facing side of the dressing 800 during shipping, storage, and handling of the dressing 800, and can be removed to expose the adhesive of the adhesive border 822 and a patient interface layer of the dressing 800 at the time of application. The removable backing 824 can be provided with flaps (folds, tabs, etc.) as shown in FIG. 8 to allow for easy removal of the removable backing 824 from the dressing 800 when the dressing is ready for application to a patient.

FIG. 8 also shows a connection port 826 positioned at the body portion 802 of the dressing 800. The connection port 826 can be positioned elsewhere on the dressing 800 in various embodiments. The connection port 826 is configured to connect the manifold layer of the dressing 800 to tubing (conduit, etc.) that extends to a negative pressure source, such that the manifold layer is in pneumatic communication with the negative pressure source and the negative pressure source can operate to establish a negative pressure at the manifold layer (e.g., by pumping air out of the manifold layer via the connection port 826 and the tubing.

Referring now to FIG. 9, a first variation of the dressing 800 is shown from a top view, according to an exemplary embodiment. In the example of FIG. 9, the adhesive border 822 extends fully across the gap 810 between the first tip 812 of the first wing 804 and the first side 808 of the body portion 802 and fully across the gap 816 between the second tip 818 of the second wing 806 and the second side 814 of the body portion 802. Accordingly, although the layers that define the dressing 800 are shown with the winged shape described above, in the example of FIG. 9 the adhesive border closes the gap 810 between the first wing 804 and the body portion 802 and the gap 816 between the second wing 806 and the body portion 802. The adhesive border can be coupled to a patient's skin across the gaps 810, 816 to facilitate sealing of the dressing 800 to a patient. In other cases, a healthcare provider may use a scissors or other tool to cut the adhesive border 822 in one or both of the gaps 810, 816 to facilitate conformance of the dressing 800 to a patient in a customizable manner.

Referring now to FIG. 10, a second variation of the dressing 800 is shown from a top view, according to an exemplary embodiment. In the example of FIG. 10, the adhesive border 822 is perforated in the gaps 810, 816. FIG. 10 shows first perforations 1000 extending along a center line of the gap 810 between the first side 808 of the body portion 802 and the first tip 812 of the first wing 804 (e.g., equally offset from the first side 808 and the first tip 812). The first perforations 1000 extend radially from a meeting point between the first wing 804 and the body portion 802. FIG. 10 also shows second perforations 1002 extending along a center line of the gap 816 between the second side 814 of the body portion 802 and the second tip 818 of the second wing 806. The second perforations 1002 extend radially from a meeting point between the second wing 806 and the body portion 802. The first perforations 1000 allow the adhesive border 822 to be torn in the first gap 810 in a controlled manner and the second perforations 1002 to be torn in the second gap 816 in a controlled manner.

When selectively torn by a caregiver, the first perforations 1000 and the second perforations 1002 allow the sizes of the first gap 810 and the second gap 816 to be adjusted to facilitate application of the dressing 800 in a desired geometry. For example, by tearing the adhesive border 822 along the first perforations 1000, the first wing can be either pulled further apart from the first side 808 of the body portion 802 (by creating space at the first perforations 1000) or pulled closer to the first side 808 of the body portion 802 (by overlapping portions of the adhesive layer from either side of the first perforations 1000). The second perforations 1002 enable similar options for adjusting the size of the second gap 816. Accordingly, the first perforations 1000 and the second perforations 1002 facilitate application of the dressing 800 to different anatomical regions or for patients of different sizes.

Referring now to FIG. 11, a third variation of the dressing 800 is shown from a top view, according to an exemplary embodiment. In the example of FIG. 11, the adhesive border 822 is formed to have a first notch 1100 at the first gap 810 and a second notch 1102 at the second gap 816. The first notch 1100 and the second notch 1102 are such that the adhesive border 822 has a shape that substantially matches the winged shape of the dressing 800. The first notch 1100 extends partially into the first gap 810, such that adhesive border 822 extends continuously across only a portion of the first gap 810. The second notch 1102 extends partially into the second gap 816, such that the adhesive border extends continuously across only a portion of the second gap 818. In some cases (e.g., for some patients, for some applications), the first notch 1100 and the second notch 1102 enable a sufficient degree of adjustability in the size of the first gap 810 and the second gap 816 (e.g., by expanding a notch, by overlapping material to reduce the notch). In other cases, the adhesive border 822 can be cut or torn between the first notch 1100 and the first wing 804 and/or the first side 808 of the body portion 802 to enable further adjustability. Similarly, the adhesive border 822 can be cut or torn between the second notch 1102 and the second wing 806 and/or the second side 814 of the dressing 800 to enable further adjustability. Accordingly, the first notch 1100 and the second notch 1102 facilitate application of the dressing 800 to different anatomical regions or for patients of different sizes.

Referring now to FIG. 12, an illustration of the dressing 800 of FIG. 8 applied to a patient in a first application location and orientation is shown, according to an exemplary embodiment. FIG. 12 shows a first perspective view 1200, a second perspective view 1202, and a third perspective view 1204 to provide a multi-angled illustration of how the dressing 800 can be applied to a patient's shoulder. The first perspective view 1200 and the second perspective view 1202 show the dressing 800 applied to the patient's right shoulder, while the third perspective view 1204 shows the dressing 800 applied to the patient's left shoulder.

To apply the dressing 800 as shown in FIG. 12, the removable backing 824 can first be removed from the dressing 800. The dressing 800 may be substantially flat (e.g., planar) as packaged and distributed. During application, the dressing 800 is bent to conform to the patient's shoulder in order to reach the application shown in FIG. 12. The body portion 802 of the dressing 800 is conformed to a lateral side of the shoulder region (e.g., an outside of the patient, over a deltoid muscle) and bent to extend over the shoulder to a superior (top) side of the shoulder region. As shown in FIG. 12, the body portion 802 is oriented such that the concave edge 820 is positioned at a superior side of the shoulder and closest to the patient's neck, with the first wing 804 and the second wing 806 also positioned extending from the body portion 802 at the superior side of the shoulder.

When the dressing 800 is applied to the right shoulder of a patient as shown in the first perspective view 1200 and the second perspective view 1202 of FIG. 12, the first wing 804 is bent (folded, etc.) relative to the body portion 802 to conform to a posterior side of the right shoulder of the patient (i.e., towards the patient's back), and the second wing 806 is bent to conform to an anterior side of the right shoulder of the patient (i.e., toward the patient's front). When the dressing 800 is applied to the left shoulder of a patient as shown in the third perspective view 1204 of FIG. 12, the first wing 804 is bent relative body portion 802 to conform to an anterior side of the left shoulder of the patient and the second wing 806 is bent to conform to a posterior side of the left shoulder of the patient. As shown in FIG. 12, the first gap 810 and the second gap 816 can be approximately aligned with an arm pit of the patient (e.g., a point, line, or area at which the patient's arm separates from the patient's torso.

The adhesive border 822 is contacted against the patient's skin to couple the dressing 800 to the patient and to provide a substantially air-tight seal between the patient and the dressing 800. In some cases, applying the dressing 800 may include adjusting the size of the first gap 810 by bending the first wing 804 toward or away from the first side 808 of the body portion 802 and using the adhesive border 822 to hold the first gap 810 in its adjusted form. For example, a first section of the adhesive border 822 may be overlapped with a second section of the adhesive border 822 at the first gap 810 to keep the first gap 810 at a reduced size. The adhesive border 822 can then also still be coupled to the patient's skin. In other cases, the first gap 810 can be widened by pulling the first wing 804 away from the first side 808 of the body portion 802 and coupling the adhesive border 822 to the patient while the gap 810 is widened to maintain the widened spacing at the gap 810. The second gap 816 can be similarly manipulated.

Applying the dressing 800 as in FIG. 12 can also include coupling the connection port 826 of the dressing 800 to a negative pressure source. The negative pressure source can then be operated to draw the dressing 800 to a negative pressure to provide negative pressure therapy at the shoulder region covered by the dressing 800. In some cases, a shoulder immobilization device (e.g., sling) may also be used to immobilize the shoulder to which the dressing 800 has been applied. In some embodiments, the negative pressure source is coupled to the shoulder immobilization device.

As illustrated in FIG. 12, the dressing 800 can be applied to provide substantially complete, continuous coverage of a shoulder region of the patient. Accordingly, the dressing 800 may cover a substantial amount of intact skin in addition to a wound or incision at the shoulder region. In some cases, the dressing 800 is even in scenarios without wounds or incisions at the skin of the patient. By providing coverage of the shoulder region and due in part to the winged shape of the dressing 800, the dressing 800 allows a large extent of the shoulder region to be exposed to a negative pressure which can cause swelling reduction at the shoulder region in addition to benefits to wound healing. Accordingly, the dressing 800 may be suitable for treating injuries (e.g., sprains, dislocations, soft tissue tears, bone breaks and fractures), for example to help reduce swelling in advance of a surgical operation, as well as post-operatively to facilitate healing of surgical incisions and promoting recovery of underlying tissue. The dressing 800 can also be used to treat burns, traumatic wounds, sores, etc. that may present at the shoulder region of the patient. The dressing 800 can thereby provide various therapeutic benefits by providing substantially complete and continuous coverage of the shoulder region.

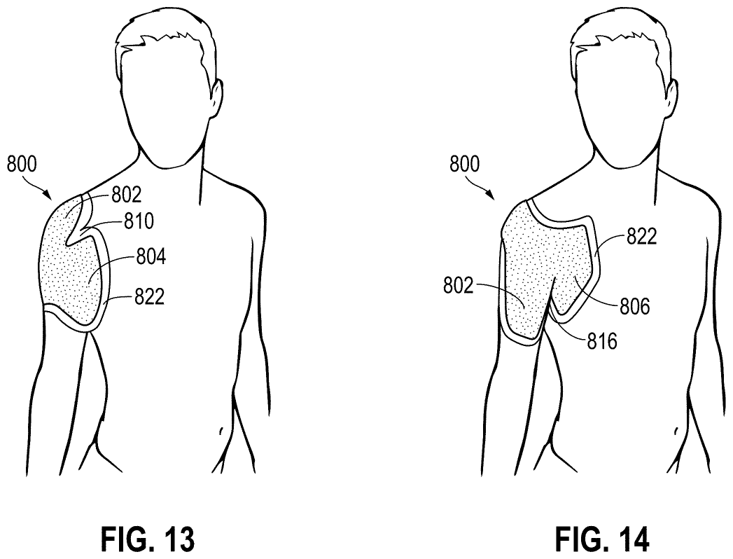

Referring now to FIG. 13, an illustration of the dressing 800 of FIG. 8 applied to a patient in a second application location and orientation is shown, according to an exemplary embodiment. FIG. 13 shows the dressing 800 applied upside-down relative to the dressing 800 of FIG. 1. FIG. 13 shows the body portion 802 extending downwards from a superior side of the shoulder region along a lateral side of the shoulder region. The first wing 804 and the second wing 806 are positioned at a bottom of the dressing 800 in the orientation shown in FIG. 13, such that the first wing 804 and the second wing 806 are positioned around the patient's deltoid and/or bicep muscles. Accordingly, in FIG. 13, the dressing 800 primarily covers the patient's upper arm and may be suitable for treating wounds on a lateral surface of the arm, for example where an incision may be made during a shoulder arthroplasty procedure. FIG. 13 thereby provides an example of the flexibility of the dressing 800 to be applied in a variety of orientations and positions as may be deemed advantageous by a caregiver.

Referring now to FIG. 14, an illustration of the dressing 800 of FIG. 8 applied to a patient in a third application location and orientation is shown, according to an exemplary embodiment. As shown in FIG. 15, the dressing 800 is applied in a similar orientation as in FIG. 13, but the dressing has been shifted downwards along the patient's arm. In particular, as shown in FIG. 14, the second wing 806 of the dressing 800 is adhered to the patient's chest (e.g., over the patient's pectoral muscle). The gap 816 is positioned at the patient's arm pit to allow the body portion 802 of the dressing to extend along the patient's arm while the first wing 804 is positioned over the patient's chest. The first wing 804 may be sealed to a lateral and/or posterior surface of the patient's arm. Accordingly, the dressing 800 is shows as providing coverage of the anterior and lateral sides of the shoulder region, including a large extent of the upper arm of the patient, while leaving the superior surface of the shoulder region exposed. Such an application may be advantageous depending on the locations of wounds or injuries to the patient's shoulder. FIG. 14 thereby provides another example of the ability of the dressing 800 to be applied in a variety of orientations and positions as may be deemed advantageous by a caregiver.

Although FIGS. 13 and 14 show alternative applications which are still at or proximate the patient's shoulder, other applications where the dressing 800 is applied to other anatomical regions are also possible. For example, the dressing 800 is advantageously shaped to be positioned on a patient's back, for example in a upper thoracic region (e.g., with the concave edge 820 facilitating conformance to the patient's neck), or a lumbar or sacral region (e.g., with the wings 804, 806 improving coverage over the patient's lower back or pelvis while the body portion 802 extends along the patient's spine. As further example, the dressing 800 can be applied to a patient's hip or elbow.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A negative pressure therapy dressing, comprising:
a drape layer;
an adhesive border configured to provide a seal between the drape layer and skin when the dressing is applied to a patient; and
a manifold layer coupled to the drape layer, wherein:
the manifold layer comprises a body portion extending in a first direction, a first wing positioned at a first side of the body portion, and a second wing positioned at a second side of the body portion opposite the first side;
the first wing extends away from the first side and partially in the first direction such that a first gap is provided between a first tip of the first wing and the first side of the body portion; and
the second wing extends away from the second side and partially in the first direction such that a second gap is provided between a second tip of the second wing and the second side of the body portion.

2. The negative pressure therapy dressing of claim 1, wherein the adhesive border extends across the gap.

3. The negative pressure therapy dressing of claim 2, wherein the adhesive border is perforated in the gap to facilitate tearing or cutting of the adhesive border in the gap.

4. The negative pressure therapy dressing of claim 1, wherein the body portion comprises a concave edge extending between the first wing and the second wing.

5. The negative pressure therapy dressing of claim 1, wherein the manifold layer is scored to facilitate conformability of the manifold layer.

6. The negative pressure therapy dressing of claim 1, wherein the manifold layer is symmetric across a longitudinal axis of the body portion.

7. The negative pressure therapy dressing of claim 1, wherein the first wing comprises an elliptical shape.

8. The negative pressure therapy dressing of claim 1, wherein the adhesive border comprises an adhesive configured to selectively adhere to the skin, the adhesive border, and the drape.

9. The negative pressure therapy dressing of claim 1, wherein the manifold layer is configured to be applied to a shoulder region of the patient, body portion corresponding to lateral and superior sides of the shoulder region of the patient, the first wing corresponding to an anterior side of the shoulder region of the patient, and the second wing corresponding to a posterior side of the shoulder region of the patient.

10. A negative pressure therapy system, comprising:
a dressing defining a sealable volume and comprising a body portion extending in a first direction and a first wing positioned at a first side of the body portion, wherein the first wing extends away from the first side and partially in the first direction such that a first gap is provided between a first tip of the first wing and the first side of the body portion; and
a negative pressure source configured to be placed in fluid communication with the dressing and operable to establish a negative pressure at the sealable volume.

11. The negative pressure therapy system of claim 10, further comprising an immobilization device configured to immobilize a shoulder of a patient, wherein the negative pressure source is coupled to the immobilization device.

12. The negative pressure therapy system of claim 10, wherein the dressing comprises an adhesive border.

13. The negative pressure therapy system of claim 12, wherein the adhesive border extends across the gap.

14. The negative pressure therapy system of claim 13, wherein the adhesive border is perforated in the gap to facilitate tearing or cutting of the adhesive border in the gap.

15. The negative pressure therapy system of claim 10, wherein the dressing further comprises a second wing positioned at a second side of the body portion, wherein the second wing extends away from the first side and partially in the first direction such that a second gap is provided between a second tip of the second wing and the second side of the body portion.

16. The negative pressure therapy system of claim 15, wherein the body portion comprises a concave edge extending between the first wing and the second wing.

17. The negative pressure therapy system of claim 15, wherein the dressing is symmetrical across a longitudinal axis of the body portion.

18. The negative pressure therapy system of claim 10, wherein the first wing comprises an elliptical shape.

19. A method of providing negative pressure therapy, comprising:
positioning a body portion of a dressing along a lateral side of a joint region of a patient;
bending the dressing to position a first wing of the dressing along an anterior side of the joint region of the patient;
bending the dressing to position a second wing of the dressing along a posterior side of the joint region of the patient;
adjusting a first gap between the body portion and the first wing and a second gap between the second wing and the body portion to conform the dressing to the joint region;
establishing a substantially air-tight seal between the dressing and the joint region using an adhesive border of the dressing;
coupling the dressing to a negative pressure source; and
operating the negative pressure source to establish a negative pressure at the dressing and the joint region.

20. The method of claim 19, wherein the joint region comprises at least one of a shoulder, elbow, hip, knee, or ankle of the patient:
creating a cut or tear in the adhesive layer proximate the first gap; and
overlapping the adhesive layer at the cut or tear such that the adhesive layer self-adheres to maintain an adjustment to the first gap.

21. The method of claim 19, wherein adjusting the first gap comprises:
creating a cut or tear in the adhesive layer proximate the first gap; and
overlapping the adhesive layer at the cut or tear such that the adhesive layer self-adheres to maintain an adjustment to the first gap.

22. The method of claim 21, wherein adjusting the first gap comprises decreasing a distance between a tip of the first wing and a first side of the body portion.

23. The method of claim 19, further comprising immobilizing the joint region using an immobilization device; and
coupling the negative pressure device to the immobilization device.
